(19) United States
(12) Patent Application Publication
(10) Pub. No.: US 2008/0119938 A1
(43) Pub. Date: May 22, 2008

(54) Knee joint prosthesis for bi-compartmental knee replacement and surgical devices thereof

(76) Inventor: Sang Soo Oh, Chenju (KR)

Correspondence Address: JENKINS, WILSON, TAYLOR & HUNT, P. A., 3100 TOWER BLVD., Suite 1200, DURHAM, NC, 27707, US

(21) Appl. No.: 11/809,304

(22) Filed: May 31, 2007

(30) Foreign Application Priority Data
Nov. 17, 2006 (KR) 10-2006-0113737
Mar. 8, 2007 (KR) 10-2007-0022798

(51) Int. Cl. A61F 2/38

(52) U.S. Cl. 623/20.14; 623/20.19; 623/20.21; 623/20.23

(57) ABSTRACT

There are provided a knee joint prosthesis for a bi-compartmental knee replacement and surgical devices thereof, wherein the knee joint prosthesis for the bi-compartmental knee replacement, which is performed on a patient with degenerative arthritis at any one of an inside compartment and an outside compartment of the knee and between the femur and the patella, is configured to position a femoral component at the inside or outside of the femur of the patient, to position a tibial component at the inside or outside of the tibia, and to properly position a tibial bearing member between the femoral component and the tibial component, thereby having the effects of improving the range of sense and motion after surgery by preserving other normal joints and reducing blood loss compared to a conventional prosthesis; improving the accuracy of the surgery to prevent the malalignment of the prosthesis, reduce the shaking of the prosthesis and extend the use life of the prosthesis; significantly improving a securing force compared to a conventional prosthesis; and using the knee joint prosthesis for the bi-compartmental knee replacement for patients with bi-compartmental arthritis, genu varum and genu valgum and flexion deformity, and anterior cruciate ligament loss.

### CROSS-REFERENCE TO RELATED PATENT APPLICATION

This application claims the benefit of Korean Patent Application No. 10-2006-0113737, filed on Nov. 17, 2006 and 10-2007-0022798, filed on Mar. 8, 2007 in the Korean Intellectual Property Office, the disclosure of which is incorporated herein in its entirety by reference.

### BACKGROUND OF THE INVENTION

1. Field of the Invention

The present invention relates to a knee joint prosthesis for a bi-compartmental knee replacement and surgical devices thereof, and more particularly, to a knee joint prosthesis for a bi-compartmental knee replacement which is performed on a patient with degenerative arthritis at any one of an inside compartment and an outside compartment of the knee and between the femur and the patella, and surgical device thereof. When using the knee joint prosthesis for the bi-compartmental knee replacement, a femoral component is positioned at any one of the inside and outside of the femur of the patient, the front part and the entire front corner part thereof; a tibial component is positioned at any one of the inside and outside of the tibia of the patient; and a tibial bearing member is properly positioned between the femoral component and the tibial component. Therefore, the knee joint prosthesis for the bi-compartmental replacement prevents the range of motion and proprioception from decreasing after the surgery and reduces surgery time and costs, compared to a conventional knee prosthesis for a total knee replacement used by removing the other normal joints when the surgery is performed on the patient with degenerative arthritis present at only any one of the inside and outside compartments of the knee. Further, since the knee joint prosthesis for the bi-compartmental knee replacement is used by preserving the normal joints upon the surgery, the flexion gap-extension gap and the ligament balance are easily adjusted, so that the malalignment of the prosthesis is prevented and the wear debris thereof is reduced, extending the use life of the prosthesis.

2. Description of the Related Art

Joint replacement surgery has been operated very general. Many people who are unable to normally do their activities before surgery are able to resume their normal activities after surgery. Artificial joints are made of metals, ceramic and/or plastic materials to be secured to the present bones.

The knee arthroplasty is well-known surgery to replace a damaged or diseased knee joint with a prosthetic knee joint. A typical knee prosthesis includes a femoral component, a patellar component, a tibial tray or a tibial plateau, and a tibial support member. In general, the femoral component includes a pair of condyles which are spaced apart from each other laterally. The end surface of the femoral component is connected to complementary condylar elements which are formed in the tibial support member in the form of a joint.

Further, in order for an artificial knee joint to properly act, the condylar area of the femoral component should be capable of doing free sliding and rolling operations over the joint surface formed of the condylar elements of the tibial support member. The natural friction of the replaced artificial joint removes particles of debris (i.e., metals or plastics separated from the prosthesis), thereby increasing the wear debris moving in the joint. The wear debris of the artificial joint interrupts a proper mechanical function of the artificial joint.

Moreover, the wear debris may cause a bone to be broken and damaged. When the wear debris increases in the artificial joint, sometimes the surgery is needed to remove the debris or to replace the artificial joint. Then, while a properly implanted prosthetic knee joint is normally used, load and stress are applied to the tibial support member.

Generally, the knee joint is generally divided into three compartments, that is, an inside compartment between the inside femur and the inside tibia, an outside compartment between the outside femur and the outside tibia, and a joint compartment between the femur and the patella. Generally, many patients have bi-compartmental degenerative arthritis. Bi-compartmental degenerative arthritis is caused when the cartilage of the inside compartment or the outside compartment and the cartilage of the femur-patella joint are worn out. At present, the knee arthroplasty for treating bi-compartmental degenerative arthritis is typically performed by a tri-compartmental knee replacement for replacing the inside compartment, the outside compartment and the patella, and a uni-compartmental knee replacement is performed by replacing only one compartment. The total knee replacement is performed on a patient with degenerative arthritis which is present at only any one of the inside compartment and the outside compartment of the knee of the patient. When the knee prosthesis is used for the total knee replacement, the other normal compartments are removed. Accordingly, soft tissues are damaged and blood loss increases. Moreover, the prosthesis used for the total knee replacement has the difficult in adjusting the flexion-extension gap and the ligament balance upon the total knee replacement surgery, compared to the uni-compartmental knee replacement surgery performed without removing the other normal compartments. As a result, the prosthesis is likely to be misaligned and shaken, and the use life of the prosthesis is shortened. Further, when the uni-compartmental knee replacement is performed on the patient with degenerative arthritis which is present at any one of the inside compartment and the outside compartment of the knee of the patient, a prosthesis is used without removing the other normal compartments. However, the prosthesis for the uni-compartmental knee replacement is weak in a securing force. Moreover, the prosthesis for the uni-compartmental knee replacement cannot be used for the patient with bi-compartmental degenerative arthritis, the patient with severe deformity, such as genu varum and genu valgum, or flexion deformity, and the patient with anterior cruciate ligament loss. Moreover, since the bone deficit of the tibia at which the prosthesis is positioned is severe, there are considerable difficulties upon re-replacement surgery. Moreover, the prosthesis for the uni-compartmental knee replacement is less used because its use life is shorter, compared to the prosthesis of the total knee replacement.

Further, the tibial support member is made of ultra high molecular weight poly ethylene. Friction, continuous circulation and stress would cause some wear and debris of the tibial support member, resulting in wear debris, i.e., wear debris caused by Inaccurate and incomplete implantation of the prosthesis or by normal use thereof. Upon malalignment, the load applied to the tibial support member is not evenly distributed. Rather, the excessive load acts on a certain area of the tibial support member. The uneven distribution of the load (or edge load) can accelerate the increase of the wear debris. The contact stress to the tibial support member is substantially increased by the malalignment of the joint. Therefore, when the prosthetic knee joint is misaligned, the risk of generating wear debris increases.

### SUMMARY OF THE INVENTION

The present invention provides a knee joint prosthesis for a bi-compartmental knee replacement performed on a patient with degenerative arthritis at any one of an inside compartment and an outside compartment of the knee and between the femur and the patella, in which a femoral component is positioned at any one of an inside and an outside of the femur of the patient, a front part and the entire front corner part; a tibial component is positioned at any one of an inside and an outside of the tibia of the patient; and a tibial bearing member is positioned between the femoral component and the tibial component. Since lower ends and back parts of the inside and outside of a normal joint which performs the most important function in sliding and rolling operations as the basic motion of a knee joint are preserved, the range of sense and motion is improved. Since only the part of the knee joint which is damaged by arthritis undergoes the surgery, preserving the normal knee joint, a ligament balance and flexion-extension gaps are easily controlled, thereby preventing malalignment or shaking of the knee joint caused by a surgical error.

The present invention also provides a knee joint prosthesis for a bi-compartmental knee replacement, in which a contact area with a bone to be cut is wide, one peg for securing the prosthesis to the bone is positioned at a lower end of the prosthesis, and the other peg is positioned at a front corner part to be opposite to the lower end thereof, thereby increasing the adhesiveness and securing force; in which a smooth joint curve surface is formed so as not to be raised where the normal joint surface contacts with the prosthesis in the front corner part of the femur and so as to be progressively thicker from the end; and in which a tibial prosthesis is connected after removing the anterior cruciate ligament, cutting the surface of the tibial up to a front part of the posterior cruciate ligament on the extension line of the intertibial spine inside the tibial tuberosity, and cutting the inside or outside tibial condyle damaged by arthritis. Therefore, the tibial prosthesis is deeply held in the tibia like the total knee replacement, and since the keel is formed in a corrugated wing shape, the adhesiveness is more reinforced. Furthermore, since the bone deficit is less, re-replacement surgery is easy.

The present invention also provides a knee joint prosthesis for a bi-compartmental knee replacement, in which a front part of an upper surface of the tibial bearing member in a concave (deep dish) shape is higher than a back part thereof, so as to prevent shaking of the front part which occurs since the anterior cruciate ligament is removed. Therefore, the knee joint prosthesis improves the stability of a joint. Further, when arthritis is present between the femur and the patella, the bi-compartmental knee replacement is done by attaching the patella component after cutting the patella.

The present invention also provides surgical devices of the knee joint prosthesis for the bi-compartmental knee replacement performed on a patient with degenerative arthritis in any one of the inside compartment and the outside compartment and between the femur and the patella, which enables surgery to be performed for only any one of the inside compartment and the outside compartment of the knee while preserving the normal joint of the patient. Therefore, the bi-compartmental knee replacement is easily and accurately performed.

A knee joint prosthesis for a bi-compartmental knee replacement and surgical devices thereof according to the embodiments of the present invention have the following characteristics.

According to an aspect of the present invention, there is provided a knee joint prosthesis for a bi-compartmental knee replacement, which is used for metical treatment for a patient with degenerative arthritis present any one of an inside compartment and an outside compartment and between the femur and the patella, comprising: a femoral component, a tibial component and a tibial bearing member. The femoral component comprises: a femoral distal surface including pegs, wherein one peg protrudes to be fixedly positioned at a lower end part of the femur of the patient, and the other peg is positioned to be spaced apart from the peg in a direction towards a front corner part; a femoral bottom surface formed in a bending curve, so as to contact with the tibia; a femoral anterior part protruding upward, to contact with a front patella, along the femoral distal surface; and a femoral posterior part formed to be opposite to the femoral anterior part and protruding vertically and upward, along the femoral distal surface. The tibial component comprises: a tibial connection part protruding in a corrugated wing shape, so as to be inserted into the tibia of the patient, corresponding to the femoral component; and tibial plateau part formed in a single body with an upper end of the tibial connection part and including a connection groove formed on a top surface thereof. The tibial bearing member which is positioned between the femoral component and the tibial component comprises: a bearing top surface formed in a concave shape, so as to contact with the bottom surface of the femoral component, and formed in the manner that a front part of the bearing top surface is higher vertically and upward than a rear part thereof; and a bearing bottom surface including a connection member to be mechanically inserted into the connection groove of the tibial plateau part.

According to another aspect of the present invention, there is provided surgical devices for the knee joint prosthesis which is used for medical treatment for the patient with degenerative arthritis present the inside compartment or the outside compartment and between the femur and the patella and which comprises the femoral component to be positioned at the lower end part of the femur of the patient, the tibial component to be inserted into the tibia of the patient and the tibial bearing member to be positioned between the femoral component and the tibial component, comprising: a femoral sizer, a femoral cutting block, a femoral trial component, a tibial resection block, a tibial alignment guide, a tibial template, a punch guide, a keel punch, and a trial insert. Before the surgery is performed for the femur of the patient, the femoral sizer is positioned at the lower end part of the femur, to measure the size of the femur to decide the size of the femoral component. The femoral cutting block is formed to be adjusted for the inner circumference surface of the femoral component based on the size measured by the femoral sizer, thereby enabling the surgery to be performed for only any one of the inside and outside compartments of the lower end part of the femur and the front part and the front corner part of the femur. The femoral trial component includes a number of apertures formed at one side, to form a hole distal side and anterior chaffer of the femur after undergoing by the femoral cutting block, so that the femoral component is fitted into the distal part of the femur of the patient. The tibial resection block is formed to cut horizontally the section of any one of the inside and outside compartments of the patient's tibia into which the tibial component is inserted. The tibial alignment guide including one end to which the tibial resection block is attached is applied to the leg (about the fibula) of the patient, so that the tibial resection block is prevented from being shaken upon the surgery. The tibial template includes: an insertion opening formed through one side of the tibial template, for operating an insertion hole on the section of any one of the inside and outside compartments of the tibia so that the tibial component is inserted into the patient's tibia being cut by the tibial resection block; and a number of fixing apertures formed at the side where the insertion opening is formed so that the tibial template positioned on the section of the tibia is prevented from being shaken upon the surgery; and a number of connection apertures formed at one side, to be connected to a punch guide. The punch guide includes: movable pins fitted into and connected to the connection apertures of the tibial template; and a guide opening formed in the same shape as the insertion opening of the tibial template, so that the insertion opening and the guide opening are positioned at the same position when the punch guide is connected to the tibial template. The keel punch includes: a keel protrusion with the teeth of a saw formed at one side of the keel punch and inserted into the guide opening of the punch guide, for forming an insertion groove on the tibia of the patient, the keel protrusion formed in the same shape as the insertion opening and the guide opening. A number of trial inserts are formed in various sizes, for determining the size of the tibial bearing member before performing the surgery operation of the tibial bearing member, and formed in the same sectional shape as the tibial template, so as to be attached to or detached from the tibial template.

### BRIEF DESCRIPTION OF THE DRAWINGS

The above and other features and advantages of the present invention will become more apparent by describing in detail exemplary embodiments thereof with reference to the attached drawings in which:

FIG. 1 is a dissembled perspective view of a knee joint prosthesis for a bi-compartmental replacement according to an embodiment of the present invention;

FIG. 2 is a side view of a femoral component of FIG. 1;

FIG. 3 is a perspective view of the femoral component of FIG. 1;

FIG. 4 is a side view of a tibial component of FIG. 1;

FIG. 5 is a side view of a tibial bearing member of FIG. 1;

FIG. 6 is a plan view of a femoral sizer according to an embodiment of the present invention;

FIG. 7 is a side view of the femoral sizer;

FIG. 8 is a perspective view of a femoral cutting block according to an embodiment of the present invention;

FIG. 9 is a side view of the femoral cutting block;

FIG. 10 is a perspective view of a femoral trial component according to an embodiment of the present invention;

FIG. 11 is a side perspective view of the femoral trial component;

FIG. 12 is a perspective view of a tibial resection block according to an embodiment of the present invention;

FIG. 13 is a perspective view of a tibial alignment guide according to an embodiment of the present invention;

FIG. 14 is a side view of the tibial alignment guide;

FIG. 15 is a perspective view of a tibial template according to an embodiment of the present invention;

FIG. 16 is a perspective view of a punch guide according to an embodiment of the present invention;

FIG. 17 is a perspective view of a keel punch according to an embodiment of the present invention;

FIG. 18 is a perspective view of the tibial template, the punch guide and the keel punch being connected together; and

FIG. 19 is a perspective view of a trial insert according to an embodiment of the present invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as being limited to the embodiments set forth herein. Rather, these embodiments are provided so that this disclosure will be thorough and complete, and will fully convey the scope of the invention to those skilled in the art.

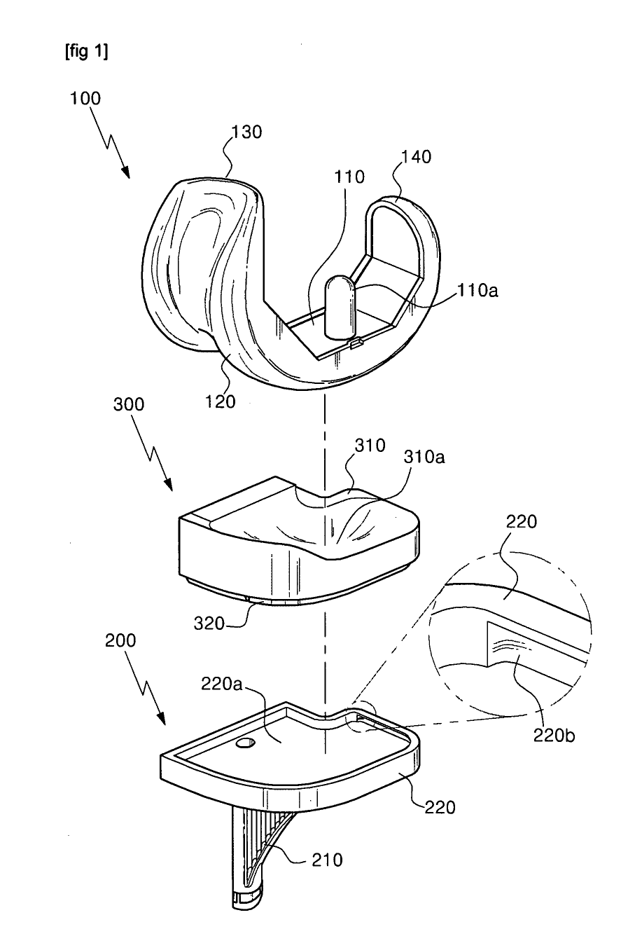

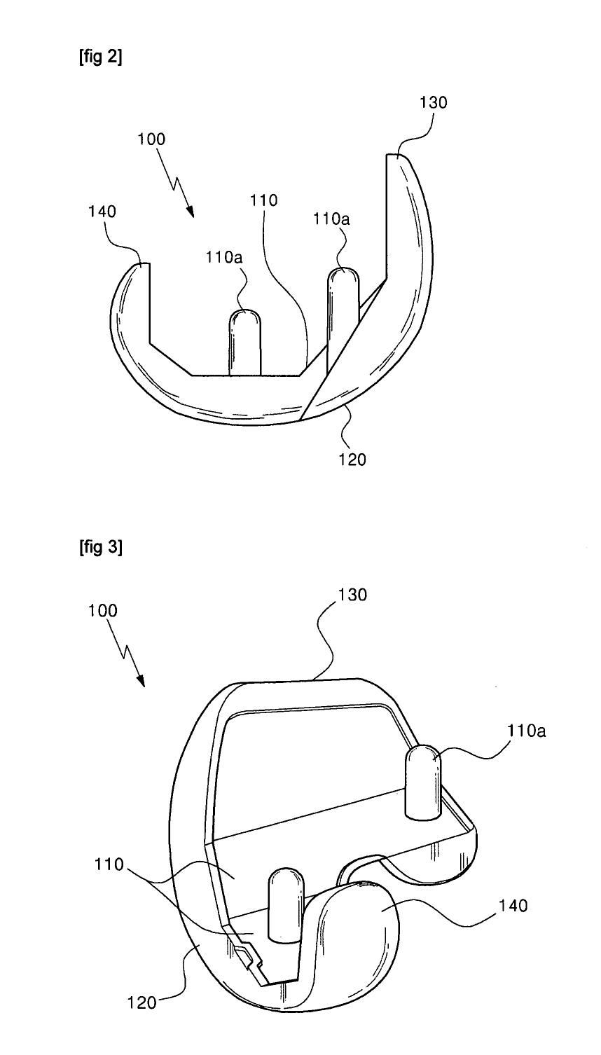

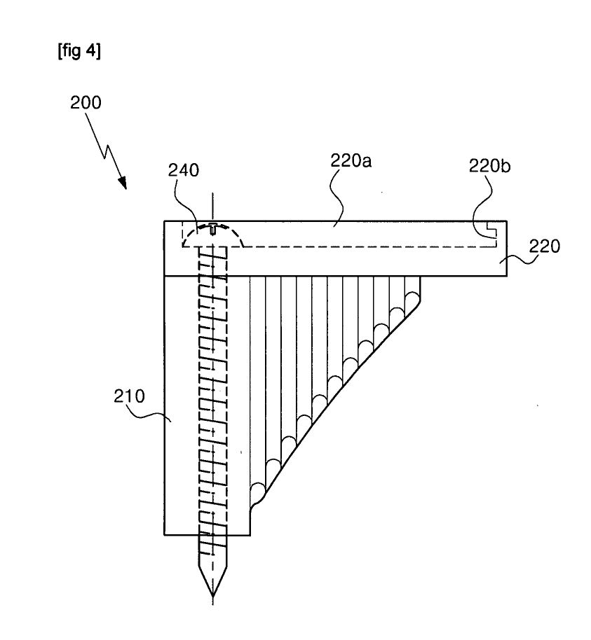

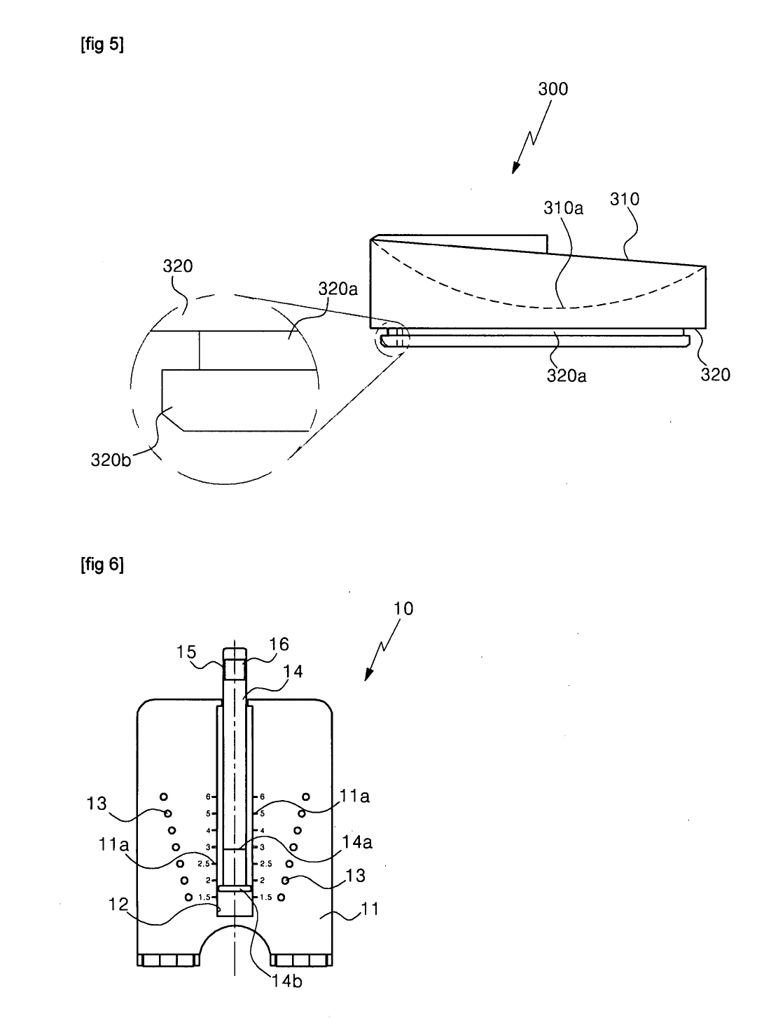

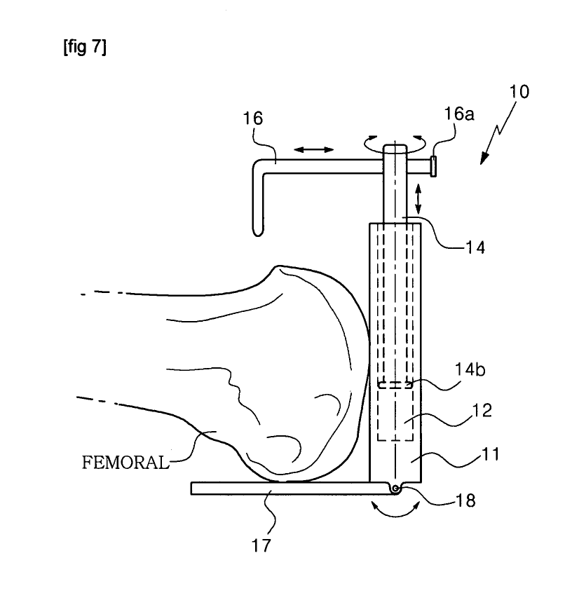

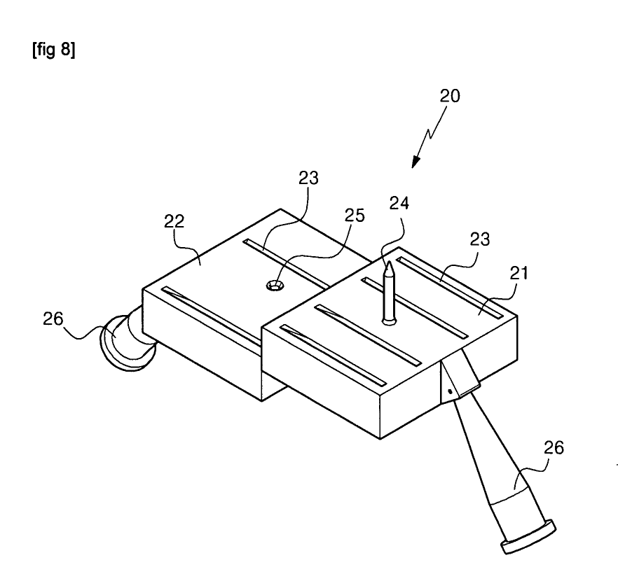

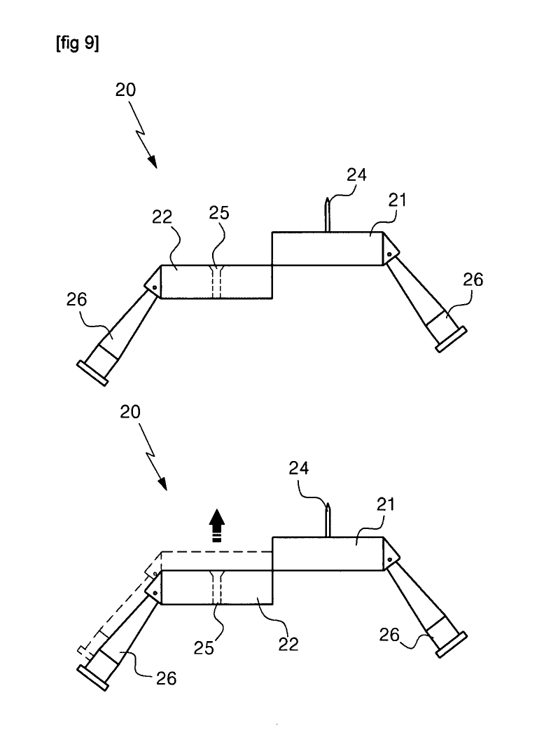

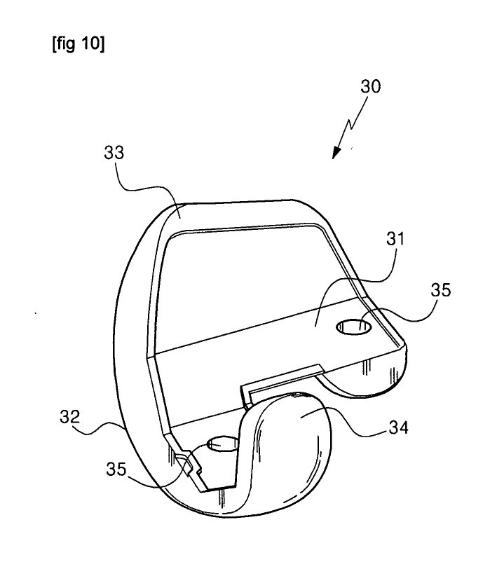

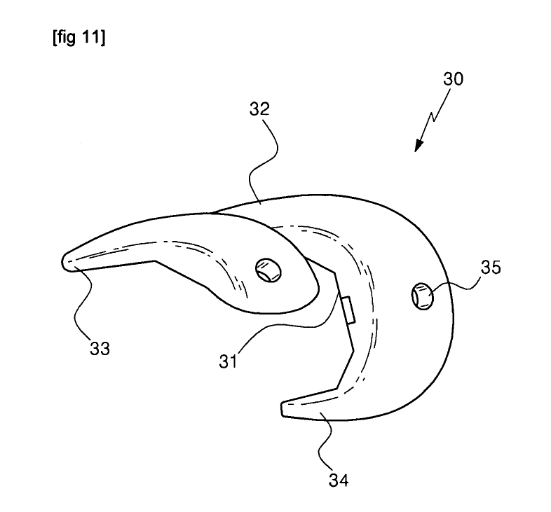

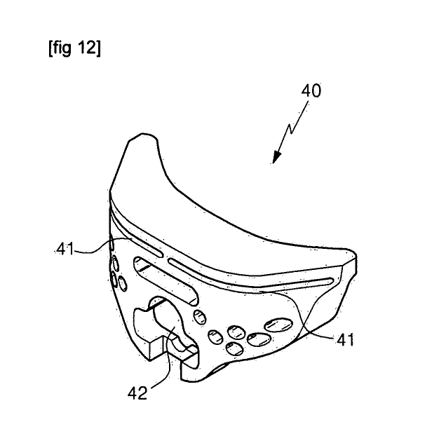

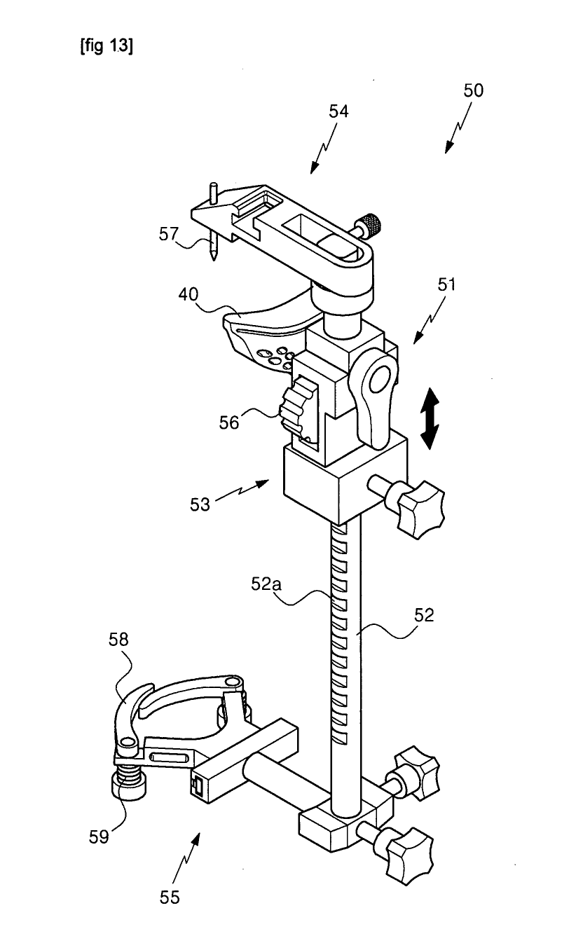

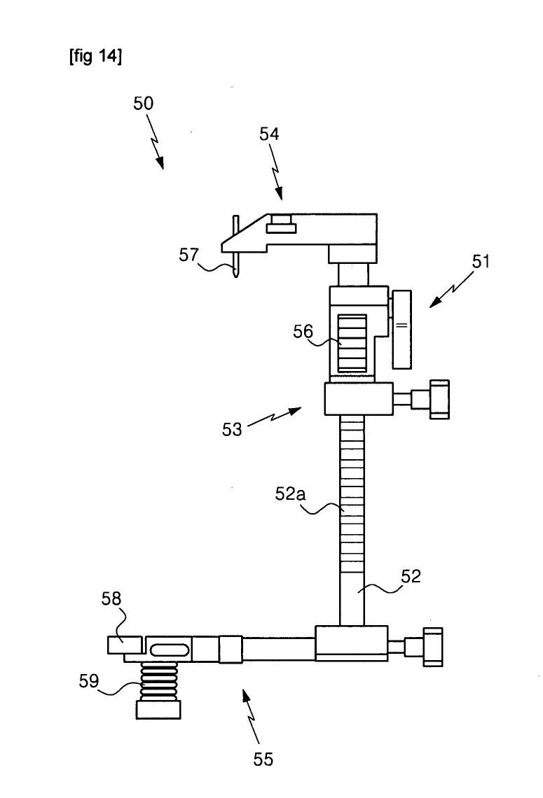

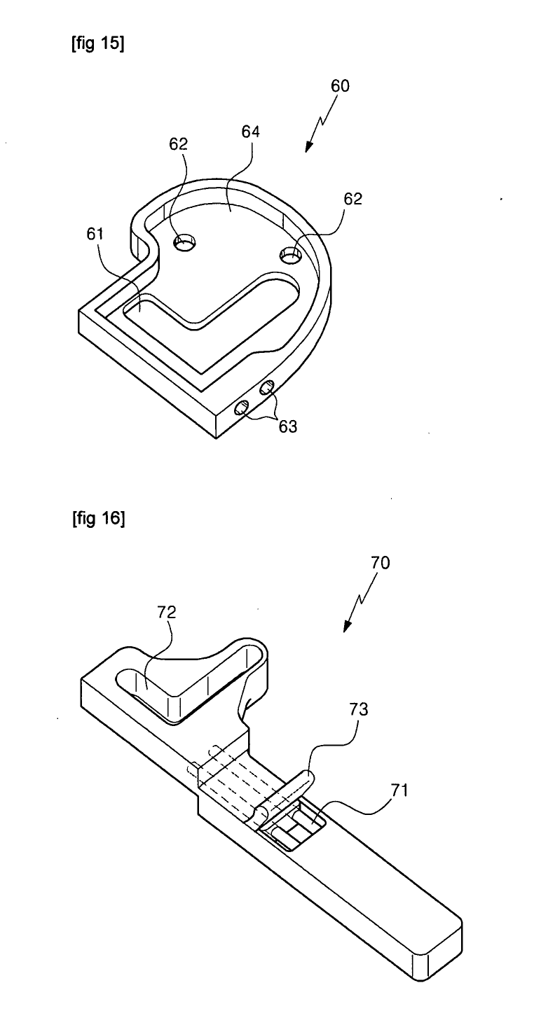

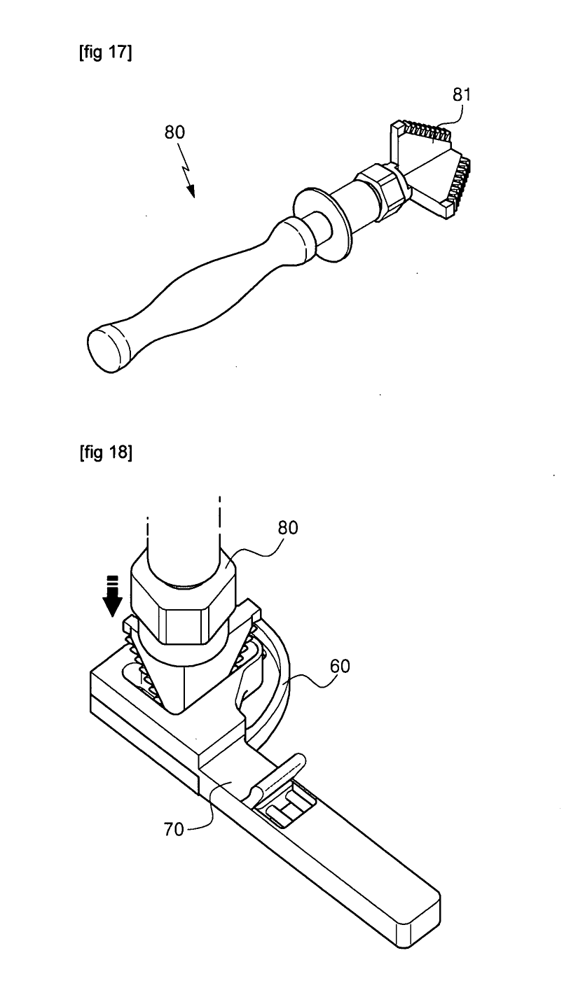

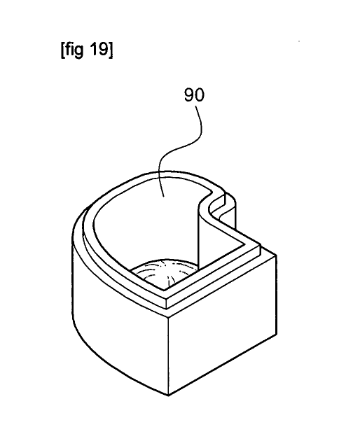

FIG. 1 is a dissembled perspective view of a knee joint prosthesis for a bi-compartmental replacement according to an embodiment of the present invention, FIG. 2 is a side view of a femoral component of FIG. 1, FIG. 3 is a perspective view of the femoral component of FIG. 1, FIG. 4 is a side view of a tibial component of FIG. 1, FIG. 5 is a side view of a tibial bearing member of FIG. 1, FIG. 6 is a plan view of a femoral sizer according to an embodiment of the present invention, FIG. 7 is a side view of the femoral sizer, FIG. 8 is a perspective view of a femoral cutting block according to an embodiment of the present invention, FIG. 9 is a side view of the femoral cutting block, FIG. 10 is a perspective view of a femoral trial component according to an embodiment of the present invention, FIG. 11 is a side perspective view of the femoral trial component, FIG. 12 is a perspective view of a tibial resection block according to an embodiment of the present invention, FIG. 13 is a perspective view of a tibial alignment guide according to an embodiment of the present invention, FIG. 14 is a side view of the tibial alignment guide, FIG. 15 is a perspective view of a tibial template according to an embodiment of the present invention, FIG. 16 is a perspective view of a punch guide according to an embodiment of the present invention, FIG. 17 is a perspective view of a keel punch according to an embodiment of the present invention, FIG. 18 is a perspective view of the tibial template, the punch guide and the keel punch being connected together, and FIG. 19 is a perspective view of a trial insert according to an embodiment of the present invention.

As illustrated in FIGS. 1 through 5, the knee join prosthesis for a bi-compartmental knee replacement comprises: a femoral component 100 positioned at one end of the femur of a patient; a tibial component 200 positioned at one end of the tibia; and a tibial bearing member 300 positioned between the femoral component 100 and the tibial component 200.

The femoral component 100 comprises: a femoral distal surface 110 contacting with an end of the femur of the patient; a femoral bottom surface 120 corresponding to the tibial component 200; a femoral anterior part 130 formed in the front of the femoral component 100, along the femoral distal surface 110; and a femoral posterior part 140 formed to be opposite to the femoral anterior part 130.

The femoral distal surface 110 is formed to be fitted into the end of the femur of the patient. A peg 110a is formed in the middle part of the femoral distal surface 110, so as to protrude upward to be secured to the femur. Another peg 110a is formed to be spaced apart from the peg 110a, towards the femoral anterior part 130. A number of the pegs 110a are inserted into the femur of the patient so that the femoral component 100 is secured.

The femoral bottom surface 120 is formed in a bending curve shape for contact with the tibial bearing member 300. The femoral bottom surface 120 is processed using cobalt-chrome and ceramic. The femoral anterior part 130 is formed at the front of the femoral component 100, along the femoral distal surface 110 and the femoral bottom surface 120. The femoral anterior part 130 is formed in the middle part of the femoral distal surface 110 so as to tilt in a front upward direction at a constant angle at a predetermined length and thereafter to protrude vertically at the angle of 90°. Then, the outer circumferential surface of the femoral anterior part 130 contacts with or is secured to the patella.

The femoral posterior part 140 is formed at the posterior of the femoral component 100, along the femoral distal surface 110 and the femoral bottom surface 120. Like the femoral anterior part 130, the femoral posterior part 140 is formed in the middle part of the femoral distal surface 110 so as to tilt in a rear upward direction at a constant angle at a predetermined length and thereafter to protrude vertically at the angle of 90°. Then, the protruding length of the femoral anterior part 130 is relatively longer than that of the femoral posterior part 140.

The femoral component 100 is formed of a half of a conventional prosthesis for a total knee replacement, so that it is positioned at only any one of an inside compartment and an outside compartment in a plan sectional view of the femur. All corner parts of the femoral component 100 are formed in a curve shape, to minimize friction by the structure in which a femoral component and the joint surface of a cut femoral condyle are smoothly connected not to be raised to maintain the shape of a normal condyle, and to cause no damage to the femur bone during connecting the femur of the patient or no pain to the patient and to reduce the wear of the prosthesis.

The tibial component 200 comprises a tibial connection part 210 inserted into the tibia of the patient; and a tibial plateau part 220 formed in a single body with an upper end of the tibial connection part 210. When the tibial component 200 is inserted into the tibia, the anterior cruciate ligament is removed and the posterior cruciate ligament is preserved, and the tibia is removed only at any one of the inside and outside undergoing the surgery.

The tibial connection part 210 is formed to protrude in a corrugated wing shape to be inserted into the tibia of the patient and has a hollow inside. A bolt 240 passes through the hollow inside of the tibial connection part 210, to secure the tibial component 200 to the tibia of the patient. Then, since the tibial connection part 210 is formed in the corrugated wind shape, the adhesiveness is more enforced when the tibial connection part 210 is inserted into the tibia. Further, since the bone deficit is less, surgery is easy upon knee re-replacement.

The tibial plateau part 220 is formed in the single body with the upper end of the tibial connection part 210 and includes a connection opening 220a formed on the top surface thereof. A connection groove 220b is formed on the inner circumferential surface of the connection opening 220a, to be connected to the tibial bearing member 300.

The tibial component 200 is characterized in that it is formed of the half of the conventional prosthesis for the total knee replacement, so that it is positioned at only any one of the inside compartment and the outside compartment in the plan sectional view of the tibia.

The tibial bearing member 300 comprises: a bearing top surface 310; and a bearing bottom surface 320. The tibial bearing member 300 is positioned between the femoral component 100 and the tibial component 200 and the bearing top surface 310 contacts with the femoral bottom surface 120 of the femoral component 100. The bearing bottom surface 320 is connected to the tibial component 200.

The bearing top surface 310 includes a contact surface 310a formed in a concave shape. The contact surface 310a is curved at the front, rear, middle and both sides thereof so as to be in contact with the femoral bottom surface 120 of the femoral component 100. The concave surface of the contact surface 310a of the bearing top surface 310 is changed in shape so as to be fitted into the curved surface formed in the femoral bottom surface 120 of the femoral component 100. Then, the front of the contact surface 310a is bent to be relatively longer upward than the posterior of the contact surface 310a, so that the femur is prevented from being bent forward.

The bearing bottom surface 320 includes a connection member 320a and a connection protrusion 320b. The connection member 320a is formed so as to be connected to the connection opening 220a formed on the top surface of the tibial component 200. The connection protrusion 320b is formed outward on the outer circumferential surface of the connection member 320a, so as to be mechanically inserted into the connection groove 220b formed in the inner circumferential surface of the connection opening 220a.

The tibial bearing member 300 is characterized in that it is formed of the half of the conventional prosthesis for the total knee replacement, so that it is positioned at only any one of the inside compartment and the outside compartment in the plan sectional view, depending on the femoral component 100 and the tibial component 200.

In this application, no description of a patella component used in the convention prosthesis is presented. However, to describe the patella component in brief, the patella component comprises a bottom surface including a number of protrusions formed to be attached to the patella, and a top surface formed to be convex to contact with the femoral component.

The above-described knee joint prosthesis for the bi-compartmental replacement according to the embodiment of the present invention is made of various biocompatible materials with high strength, durability and wear debris resistance. Examples of the materials are cobalt-potassium alloy, cobalt-chrome alloy, titanium-aluminum-vanadium alloy, stainless steel, ceramic and implantable bone prostheses. In general, the femoral component 100 is made of cobalt-potassium (chrome) or ceramic, the tibial component 200 is made of a metal alloy, such as titanium alloy, and the tibial bearing member 300 is made of a polymer, such as ultra high molecular weight poly ethylene.

Surgical devices of the knee joint prosthesis for the bi-compartmental knee replacement according to an embodiment of the present invention include: a femoral sizer; a femoral cutting block, a femoral trial component, a tibial resection block, a tibial alignment guide, a tibial template, a punch guide, a keel punch, and a trial insert. The surgical devices will be described with reference to FIGS. 6 through 19.

FIG. 6 is a plan view of a femoral sizer 10 according to an embodiment of the present invention, and FIG. 7 is a side view of the femoral sizer.

Referring to FIGS. 6 and 7, before the femoral component is operated on the femur of the patient, the femoral sizer 10 is positioned at the lower end part of the femur and measures the size of the femur to determine the femoral component. The femoral sizer 10 comprises a body 11, a movable unit 14, a measurement unit 16 and a support unit 17, to measure inside and outside, front and rear, and left and right diameters of the femur of the patient and to determine the size of the femoral prosthesis.

The femoral sizer 10 to decide the size of the femoral prosthesis before the surgery of the femur of the patient is started is formed in a “ [symbol] ” shape and is inserted between the front surface and the posterior surface of the femur.

The body 11 is supported at the lower end part of the femur. The body 11 includes a number of measurement graduations 11a for representing the size of the femur, a measurement groove 12 in a cylindrical shape formed in the center of the body, and a number of guide pin apertures 13 formed to be spaced apart from the measurement graduations 11a at predetermined intervals.

The guide pin apertures 13 are symmetrically formed at the right and left sides and formed in a multi-level shape from the outside to the inside (see FIG. 6). The guide pin apertures 13 formed at each level are positioned at the same distances from the vertical center line of the body 11 but the guide pin apertures 13 formed at the vertical line are positioned to be slightly different from one another at about 3 degrees of external rotation.

When the size of the femoral component is decided, a nail (not shown) corresponding to the size of the femoral component is inserted in each of the guide pin apertures 13. When the opposite side of the femur needs to undergo the surgery, the femoral sizer 10 is turned over and positioned so that the guide pin apertures 13 externally rotated at 3 degrees at the opposite side may be used.

The movable unit 14 is inserted into the measurement groove 12 of the body 11 and slides up and down to measure the femur. The movable unit 14 formed in the cylindrical shape and inserted into the measurement groove 12 is rotated at 360°. An indication line 14a indicating the measurement graduation 11a of the body 11 is formed at one end of the movable unit 14.

An opening 15 in a regular square shape is formed at one end of the movable unit 14. The measurement unit 16 is formed to pass through the opening 15. A support protrusion 14b is formed at the outer circumference surface of the lower end of the movable unit 14 and is inserted into the measurement groove 12 of the body 11, to prevent the movable unit 14 from being completely separated from the body 11 outward when the movable unit 14 slides up and down.

The measurement unit 16 is mounted to pass through the opening 15 formed at one end of the movable unit 14. One end of the measurement unit 16 is bent at a right angle to measure the side of the femur by the rotation of the movable unit 14. The measurement unit 16 slides back and forth to measure the side of the femur, depending on the size of the femur (the height of the side of the femur).

A connection projection 16a is formed along the outer circumferential surface of the measurement unit 16 so that the measurement unit 16 is prevented from being completely separated when the end of the measurement unit 16 passing through the opening 15 of the movable unit 14 slides back and forth.

The support unit 17 is attached at the end of the body 11 and protrudes from the end thereof opposite to the measurement unit 16, at a predetermined distance. The support unit 17 supports the femur and is connected to the body 11 by a hinge 18, so as to measure the opposite side of the femur.

In the tri-compartmental knee replacement, a conventional femoral sizer is positioned after the lower end part of the femur is cut. However, unlike the tri-compartmental knee replacement, in the bi-compartmental knee replacement, the femoral sizer 10 is positioned before the lower end part of the femur is cut (prior to the surgery). When the conventional femoral sizer is used, the measurement is inaccurate because it does not reach the apex of the posterior surface of the femur. To complement the defect of the conventional femoral sizer, in the femoral sizer 10, the length of the support unit 17 is constituted to be longer about 10 mm than that (about 20 mm) of the conventional femoral sizer.

The support unit 17 is connected to the body 11 by the hinge 18 so as to be rotated at 180°. Therefore, the femoral sizer 10 is turned over to be used when the opposite side of the femur undergoes the surgery.

FIG. 8 is a perspective view of a femoral cutting block 20 according to an embodiment of the present invention, and FIG. 9 is a side view of the femoral cutting block 20.

As illustrated in FIGS. 8 and 9, the femoral cutting block 20 is formed to perform the surgery for any one of the inside compartment and outside compartment of the lower end part of the femur of the patient, to be fitted into the inner circumferential surface of the femoral component based on the size measured by the femoral sizer 10.

The femoral cutting block 20 comprises a first body 21 and a second body 22. The first body 21 includes a femoral fixing pin 24 and a number of cutting member insertion grooves 23. The femoral fixing pin 24 formed at one surface of the first body 21 secures the femoral cutting block 20 to the femur of the patient to undergo the surgery. The cutting member insertion grooves 23 are formed in a direction of the length of the first body 21, so as to receive cutting members (mechanical saw blades) for cutting the lower end part of the femur where the femoral component is positioned. The cutting member insertion grooves 23 are spaced apart from one another at a predetermined interval. The second body 22 contacts with the normal femur of the patient by connecting to the side of the first body 21 so as to slide up and down and includes a pin opening 25 on one side thereof. The pin opening 25 is fixed to the normal femur by a pin (not shown). One end of the pin opening 25 is formed in a funnel or beaker shape so that the pin is easily inserted into the pin opening 25.

The first body 21 and the second body 22 in a single body may be formed on the same line in the manner the first body 21 is positioned at an upper level and the second level body 22 is positioned at a lower level centering the same line in a sectional view, or the first body 21 and the second body 22 in a single body are formed on the same line by sliding. Handles 26 are respectively formed at the ends of the first body 21 and second body 22. A surgeon holds the handles 26 to prevent shaking occurring upon the surgery of cutting the femur. A fixing pin hole (not shown) is formed at both sides of the first body 21 and the second body 22.

FIG. 10 is a perspective view of a femoral trial component 30 according to an embodiment of the present invention, and FIG. 11 is a side perspective view of the femoral trial component.

As illustrated in FIGS. 10 and 11, the femoral trial component 30 includes apertures 35. One aperture 35 is formed at one end of the femoral trial component 30, to form a hole at the distal surface of the femur of the patient so as to insert the femoral component peg into the distal surface of the femur after cutting the distal surface of the femur by the femoral cutting block 20. The other aperture 35 is formed at one anterior chamfer part so as to be opposite to the aperture 35 formed at one end of the femoral trial component 30. The hole for receiving the femoral component peg is formed on the lower end part of the femur, by using external mechanical equipment, such as a drill, passing through the apertures 35.

The femoral trial component 30 includes an upper surface 31, a lower surface 32, a front part 33 and a rear part 34. The upper surface is bent at various angles to contact with the lower end part of the femur of the patient. The lower surface 32 is formed in a bending curve to contact with the tibial bearing member. The front part 33 protrudes upward to contact with the front patella, along the upper surface 31. The rear part 34 is formed to be opposite to the front part 33 and protrudes upward vertically, along the upper surface 31.

Since a number of the apertures 35 through the upper surface 31 and the lower surface 32 are formed in the femoral trial component 30, the surgeon performs the surgery for the front part, front corner part, lower end part, rear corner part and rear part in any one of the inside compartment and the outside compartment of the femur of the patient and for the front part and front corner part only in the other compartment.

FIG. 12 is a perspective view of a tibial resection block according to an embodiment of the present invention.

As illustrated in FIG. 12, the tibial resection block 40 is formed to cut horizontally the section of any one of the inside compartment and the outside compartment of the patient's tibia into which the tibial component is inserted.

The front part of the tibial resection block 40 to contact with the side of the tibia is formed to be bent. The tibial resection block 40 includes a through-opening 41. The through-opening 41 is formed horizontally to receive a cutting member (mechanical saw blade) for cutting an upper part of the tibia where the tibial component needs to be positioned. The through-opening 41 is divided into two sides by its middle part which discontinues the through-opening 41, so that it enables the surgery for only any one of the inside compartment and the outside compartment of the tibia of the patient. The lower end of the tibial resection block 40 includes a connection opening 42 to be connected to a tibial alignment guide.

FIG. 13 is a perspective view of a tibial alignment guide according to an embodiment of the present invention, and FIG. 14 is a side view of the tibial alignment guide.

As illustrated in FIGS. 13 and 14, the tibial alignment guide 50 is formed to be attached to the leg (about the fibula) of the patient, to prevent the shaking of the tibial resection block 40 with its one end being attached to the tibial alignment guide 50, upon the surgery.

The tibial alignment guide 50 comprises a first fixation unit 54 which includes a connection unit 51, an axial bar 52, a control unit 53 and a second fixation unit 55. The connection unit 51 which contacts with one end of the tibial resection block 40 is connected to the connection opening 42 of the tibial resection block 40 by a connection member (bolt, pin, and the like). The axial bar 52 which passes through the inside of the connection unit 51 includes a saw tooth part 52a formed at one end thereof in a length direction. The control unit 53 includes a control lever 56 formed at the side thereof. The control lever 56 is connected to the saw tooth part 52a, so that the connection unit 51 is accurately controlled vertically in the axial bar 52. The tibial fixing pin 57 is formed at one side end of the first fixation unit 54. The tibial fixing pin 57 is vertically positioned at the upper part of the axial bar 52, to fix the tibial alignment guide 50, without shaking, upon cutting the upper part of the tibia. The second fixation unit 55 includes a number of hooks 58 to connect one side end of the tibial alignment guide 50 to the leg (about the ankle and fibula) of the patient. The hooks 58 are vertically positioned at the lower part of the axial bar 52, to fix the tibial alignment guide 50, without shaking, upon cutting the upper part of the tibia.

A spring 59 is positioned under the hooks 58 formed in the second fixation unit 55, so that a force acts in a direction of the inside of the second fixation unit 55. The hooks 58 are operated in the opposite direction to the direction of the inside of the second fixation unit 55, so as to be applied around the leg of the patient. Thereafter, the hooks 58 are returned to their original positions by the elasticity of the spring 59, to secure the leg of the patient to the tibial alignment guide 50.

FIG. 15 is a perspective view of a tibial template according to an embodiment of the present invention, FIG. 16 is a perspective view of a punch guide according to an embodiment of the present invention, FIG. 17 is a perspective view of a keel punch according to an embodiment of the present invention, and FIG. 18 is a perspective view of the tibial template, the punch guide and the keel punch being connected together.

As illustrated in FIG. 15, the tibial template 60 includes an insertion opening 61 and a number of fixing apertures 62. The insertion opening 61 is formed through one side of the tibial template 60. The insertion opening 61 function as an guide to form an insertion hole into which the tibial component is inserted on the section of any one of the inside compartment and the outside compartment of the tibia which is cut by the tibial resection block 40 to insert the tibial component. Before drilling the insertion hole, the tibial template 60 is positioned on the section of the tibia. A number of the fixing apertures 62 are formed at the side where the insertion opening 61, to prevent the shaking upon the surgery. Fixing members (kinds of pins) are nailed down through the fixing apertures 62, to prevent the tibial template 60 from being shaken.

The tibial template 60 further comprises a number of connection apertures 63 and a connection opening 64. The connection apertures 63 are formed at one end of the outer circumferential surface of the tibial template 60, so as to be connected to the punch guide 70 laterally. The axial bar (not shown) may be positioned using the connection apertures 63. The connection opening 64 is formed at one side of the tibial template 60, so as to receive the trial insert 90.

As illustrated in FIG. 16, the punch guide 70 comprises a number of movable pins 71, a guide opening 72 and a movable lever 73. A number of movable pins 71 are formed to be combined with the connection apertures 63 of the tibial template 60. The guide opening 72 is formed in the same shape as the insertion opening 61 of the tibial template 60. The guide opening 72 formed in one side of the punch guide 70 is positioned at the same position as the insertion opening 61 when the punch guide 70 is connected to the tibial template 60.

The movable pins 71 are formed inside the punch guide 70 in a direction of the length of the punch guide 70. One end of each movable pin 71 is inserted into each fixing aperture 62 of the tibial template 60. When the movable pin 71 is inserted into the fixing aperture 62, the movable pin 71 is moved back and forth by the movable lever 73 formed on the upper part of the punch guide 70 and connected to the outer circumferential surface of the movable pin 71.

As illustrated in FIGS. 17 and 18, the keel punch 80 comprises a keel protrusion 81. The keel protrusion 81 with the teeth of a saw is formed at one side of the keel punch 80. The keel protrusion 81 is inserted into the guide opening 72 of the punch guide 70, to form an insertion hole on the tibia of the patient. The keel protrusion 81 is formed in the same sectional shape as the insertion opening 61 and the guide opening 72.

When the keel protrusion 81 is inserted into the insertion opening 61 and the guide opening 72, the keel punch 80 forms the insertion hole on the upper part of the tibia at a predetermined interval, by applying an impact vertically to the other parts, using a medical mallet.

Since the shape of the keel protrusion 81, the insertion opening 61 and the guide opening 72 has an “L” shaped section, the insertion part of the tibial component is prevented from being damaged or separated by shaking or impact when the tibial component is inserted.

FIG. 19 is a perspective view of a trial insert 90 according to an embodiment of the present invention.

As illustrated in FIG. 19, a number of the trial inserts 90 are provided in various sizes, to determine the size of the tibial bearing member before the tibial bearing member is operated. The trial insert 90 is formed in the same sectional shape as the tibial template 60 so as to be attached to and detached from the tibial template 60.

The trial insert 90 is made of a rubber material and is formed in the shape of one compartment to perform the surgery for only any one of the inside compartment and the outside compartment of the knee of the patient to undergo the surgery. The trial insert 90 is formed to be the same as the surface of the bearing member contacting with the tibial component and the femoral component.

As described above, in accordance with the knee joint prosthesis for the bi-compartmental knee replacement, which is performed on the patient with degenerative arthritis at any one of an inside compartment and an outside compartment of the knee and between the femur and the patella, according to the embodiment of the present invention, the femoral component is positioned at the inside or outside of the femur of the patient, the tibial component is positioned at the inside or outside of the tibia and the front part and the entire front corner part of the tibia of the patient, and the tibial bearing member is suitably positioned between the femoral component and the tibial component. Therefore, the knee joint prosthesis for the bi-compartmental replacement according to the embodiment of the present invention prevents the range of motion and proprioception from decreasing after the surgery and reduces surgery time and costs, compared to the conventional knee prosthesis for a total knee replacement used by removing the other healthy joints upon the surgery performed on the patient with degenerative arthritis present at only any one the inside compartment and the outside compartment of the knee.

Furthermore, when the knee joint prosthesis for the bi-compartmental replacement according to the embodiment of the present invention is used, the surgery is performed by preserving the normal knee joints of the femur. Therefore, the flexion gap-extension gap and the ligament balance are easily adjusted, so that the malalignment of the prosthesis is prevented and the wear debris thereof is reduced to extend the use life of the prosthesis.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A knee joint prosthesis for a bi-compartmental knee replacement, which is used for medical treatment for a patient with degenerative arthritis present any one of an inside compartment and an outside compartment and between the femur and the patella, comprising:
a femoral component 100 comprising:
a femoral distal surface 110 including pegs 110a, wherein one peg 110a protrudes to be fixedly positioned at a lower end part of the femur of the patient, and the other peg 110a is positioned to be spaced apart from the peg 110a in a direction towards a front corner part;
a femoral bottom surface 120 formed in a bending curve, so as to contact with the tibia;
a femoral anterior part 130 protruding upward, to contact with a front patella, along the femoral distal surface 110; and
a femoral posterior part 140 formed to be opposite to the femoral anterior part 130 and protruding vertically and upward, along the femoral distal surface 110;
a tibial component 200 comprising:
a tibial connection part 210 protruding in a corrugated wing shape, so as to be inserted into the tibia of the patient, corresponding to the femoral component 100; and
tibial plateau part 220 formed in a single body with an upper end of the tibial connection part 210 and including a connection groove 220a formed on a top surface thereof; and
a tibial bearing member 300 positioned between the femoral component 100 and the tibial component 200, comprising:
a bearing top surface 310 formed in a concave shape, so as to contact with the bottom surface of the femoral component 100, and formed in the manner that a front part of the bearing top surface 310 is higher vertically and upward than a rear part thereof; and
a bearing bottom surface 320 including a connection member 320a to be mechanically inserted into the connection groove 220a of the tibial plateau part 220.

2. The knee joint prosthesis of claim 1, wherein the femoral component 100, the tibial component 200 and the tibial bearing member 300 are formed to be positioned at only any one of the inside and outside compartments of the femur and tibia in a plan section.

3. The knee joint prosthesis of claim 1, wherein the bearing upper surface 310 of the tibial bearing member 300 includes a contact surface 310a in a concave shape being bent in the front, rear, middle and both sides thereof, so that a front part of the contact surface 310a is formed to be higher upward than a rear part thereof and the contact surface 310a is formed to be fitted into the bending curve surface of the femoral bottom surface 120 of the femoral component 100.

4. Surgical devices of the knee joint prosthesis of any one of claims 1 to 3, which is used for medical treatment for the patient with degenerative arthritis present the inside compartment or the outside compartment and between the femur and the patella, comprising:
a femoral sizer 10 positioned at the lower end part of the femur before the surgery is performed for the femur of the patient, for measuring the size of the femur to decide the size of the femoral component 100;
a femoral cutting block 20 formed to be adjusted for the inner circumference surface of the femoral component 100 based on the size measured by the femoral sizer 10, so that only any one of the inside and outside compartments of the lower end part of the femur undergoes the surgery;
a femoral trial component 30 including a number of apertures 35 formed at one side to form a hole distal side and anterior chaffer of the femur after undergoing by the femoral cutting block 20, so that the femoral component 100 is fitted into the distal part of the femur of the patient;
a tibial resection block 40 formed to cut horizontally the section of any one of the inside and outside compartments of the patient's tibia into which the tibial component 200 is inserted;
a tibial alignment guide 50 including one end to which the tibial resection block 40 is attached and applied to the leg (about the fibula) of the patient, for preventing the tibial resection block 40 from being shaken upon the surgery;
a tibial template 60 including:
an insertion opening 61 formed through one side of the tibial template 60, for operating an insertion hole on the section of any one of the inside and outside compartments of the tibia so that the tibial component 200 is inserted into the patient's tibia being cut by the tibial resection block 40;
a number of fixing apertures 62 formed at the side where the insertion opening 61 is formed, for preventing the tibial template 60 positioned on the section of the tibia from being shaken upon the surgery; and
a number of connection apertures 63 formed at one side, to be connected to a punch guide 70;
a punch guide 70 including:
movable pins 71 fitted into and connected to the connection apertures 63 of the tibial template 60; and
a guide opening 72 formed in the same shape as the insertion opening 61 of the tibial template 60, so that the insertion opening 61 and the guide opening 72 are positioned at the same position when the punch guide is connected to the tibial template 60;
a keel punch 80 including:
a keel protrusion 81 with the teeth of a saw formed at one side of the keel punch 80 and inserted into the guide opening 72 of the punch guide 70, for forming an insertion hole on the tibia of the patient, the keel protrusion 81 formed in the same shape as the insertion opening 61 and the guide opening 72; and
a number of trial inserts 90 formed in various sizes, for determining the size of the tibial bearing member before performing the surgery operation of the tibial bearing member, and formed in the same sectional shape as the tibial template 60, so as to be attached to or detached from the tibial template 60.

5. The surgical devices of claim 1, wherein the femoral cutting block 20 comprises:
a first body 21 including:
a femoral fixing pin 24 formed at one side of the first body 21 to be fixed to the patient's femur to undergo the surgery;
a number of cutting member insertion grooves 23 formed in a direction of the length of the first body 21 and spaced apart from one another at predetermined interval, so that cutting members (mechanical saw blades) for cutting the femur to position the femoral component 100 are inserted; and
a second body 22 connected to the side of the first body 21 so as to slide up and down to contact with the normal femur of the patient and including a pin opening 25 formed at one side of the second body 22 to be fixed to the normal femur by a pin, and
wherein the first body 21 and the second body 22 in a single body are formed centering the same plane in the manner the first body 21 is positioned at an upper level and the second level body 22 is positioned at a lower level in a sectional view, or the first body 21 and the second body 22 in a single body slide are formed on the same line by sliding; and each of the first body 21 and second body 22 includes a handle 26 for a surgeon formed at the end of each of the first body 21 and second body 22, for preventing shaking upon the surgery of cutting the femur.

6. The surgical devices of claim 1, wherein the femoral trial component 30 comprises:
an upper surface 31 bent at various angles to contact with the lower end part of the femur of the patient;
a lower surface 32 formed in a bending curve to contact with the tibial bearing member;
a front part 33 protruding upward to contact with the front patella;
a rear part 34 formed to be opposite to the front part 33 and protruding upward vertically, along the upper surface 31; and
a number of the apertures 35 formed through the upper surface 31 and the lower surface 32 enabling the surgery to be performed for only any one of the inside and outside compartments of the femur of the patient.

7. The surgical devices of claim 1, wherein the tibial resection block 40 comprises:
a front part formed to be bent to contact with the side of the tibia;
a through-opening 41 formed through the tibial resection block 40 horizontally but having a blocking middle part, for receiving a cutting member (mechanical saw blade) for cutting an upper part of the tibia into which the tibial component 200 is positioned, and for enabling the surgery to be performed for only any one of the inside and outside compartments of the tibia of the patient; and
a connection opening 42 formed at the lower end of the tibial resection block 40, so as to be connected to the tibial alignment guide 50.

8. The surgical devices of claim 1, wherein the femoral sizer 10 is formed in a “ [symbol] ” shape to be positioned at the lower end part of the femur of the patient before the surgery and comprises:
a body 11 supported at the lower end part of the femur and including:
a number of measurement graduations 11a for representing the size of the femur;
a measurement groove 12 in a cylindrical shape formed in the center of the body 11;
a movable unit 14 inserted into the measurement groove 12 of the body 11 so as to slide up and down to measure the femur and formed in the same cylindrical shape as the measurement groove 12 so as to rotate, and the movable unit 14 including an indication line 14a formed at an end thereof to indicate the measurement graduations 11a of the body 11;
a measurement unit 16 formed through the end of the movable unit 14 and including an end which is bent at a right angle to measure the side of the femur by the rotation of the movable unit 14, and the measurement unit 16 sliding back and forth to measure the side of the femur depending on the size of the femur; and
a support unit 17 formed to protrude at a predetermined length from the end of the body opposite to the measurement unit 16 and supported by the femur, and the support unit 17 connected to the body 11 by a hinge 18, so as to measure the opposite side of the femur.

9. The surgical devices of claim 1 or claim 4, wherein the tibial alignment guide 50 comprises:
a connection unit 51 contacting with one end of the tibial resection block 40 and connected to the connection opening 42 of the tibial resection block 40 by a connection member (bolt, pin, and the like);
an axial bar 52 formed to pass through the inside of the connection unit 51 and including a saw tooth part 52a formed at one end thereof in a length direction;
a control unit 53 including a control lever 56 formed at the side thereof and connected to the saw tooth part 52a so that the connection unit 51 is accurately controlled vertically in the axial bar 52;
a first fixation unit 54 including a tibial fixing pin 57 formed at one side end thereof and vertically positioned at the upper part of the axial bar 52, so as to fix the tibial alignment guide 50, without shaking, upon cutting the upper part of the tibia; and
a second fixation unit 55 including a number of hooks 58 formed to connect one side end of the tibial alignment guide 50 and the leg (about the ankle and fibula) of the patient and vertically positioned at the lower art of the axial bar 52, so as to fix the tibial alignment guide 50, without shaking, upon cutting the upper part of the tibia.
